(19) United States
(12) Patent Application Publication
(10) Pub. No.: US 2012/0039813 A1
(43) Pub. Date: Feb. 16, 2012

(54) INTRINSICALLY COLORED, LUMINESCENT SILK FIBROIN AND A METHOD OF PRODUCING THE SAME

(76) Inventors: Natalia Chendrawati Tansil, Singapore (SG); Ming-Yong Han, Singapore (SG); Xiang Yang Liu, Singapore (SG); Radiana Soh, Singapore (SG); Yang Li, Singapore (SG)

(21) Appl. No.: 13/202,089

(22) PCT Filed: Feb. 17, 2010
(86) PCT No.: PCT/SG2010/000059

§ 371 Date: Oct. 31, 2011

Related U.S. Application Data
(60) Provisional application No. 61/153,065, filed on Feb. 17, 2009.

(51) Int. Cl. A61K 49/00; G01N 31/22; A61F 13/00; A61K 47/42; C07K 14/435; B32B 5/02; C07K 1/00; A61K 38/00

(52) U.S. Cl. 424/9.6; 435/272; 436/163; 514/1.1; 514/773; 530/350; 530/402; 428/221; 602/43

(57) ABSTRACT

This present invention proposes a method of producing intrinsically colored, luminescent silk fibroin by feeding silkworms with feed comprising the luminescent dyes as well as the appropriate degumming process to remove sericin while retaining the dyes in silk fibroin are also described. Luminescent and functional molecular organic dyes with balanced hydrophobic/hydrophobic properties that can be effectively absorbed into silk glands of the silkworm are also described.

This application makes reference to and claims the benefit of priority of an application for “Intrinsically colored, luminescent silk fibroin and the method for producing them” filed on Feb. 17, 2009 with the United States Patent and Trademark Office, and there duly assigned U.S. Provisional Ser. No. 61/153,065. The contents of said application filed on Feb. 17, 2009 is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The invention relates to a method for producing luminescent colored silk fibroin. The invention also relates to the use of luminescent colored silk fibroin to produce threads, yarns or fabrics and in biomedical applications.

### BACKGROUND OF THE INVENTION

Silk has been a highly prized material since its discovery in 2640 BC. It is tougher than cotton and warmer than wool, despite being much lighter. Even with the invention and use of manmade fibers, silk continues to enjoy a strong demand as luxury fabric. The silk industry, comprising raw silk and finished silk products, is worth an approximate USD 20 billion worldwide.

Due to its biocompatibility, silk is also widely used in the biomedical field as sutures, artificial blood vessels, and scaffolds for tissue engineering. Incorporating substances such as drugs, anti-coagulant, anti-microbial, anti-inflammatory agent, etc to these items will significantly increase their value and functionality. In most applications, only the core of silk filament (fibroin) is used while the outer gummy layer (sericin) is removed.

However, the largest portion of silk consumption comes from the textile industry, in which dyeing is an integral part of silk processing. Existing methods of silk dyeing are costly and may cause deterioration of silk properties. The complexity and high cost involved in conventional dyeing methods, such as dyeing cocoons after their formation, presents a need for alternative methods to produce colored silk.

Maintaining the superior properties of silk while adding colors (and functionalities) to silk is therefore of high commercial interest. Although genetic engineering/immersion methods report the incorporation of functional materials (e.g. dyes) into raw silk fiber, there is no clear evidence on whether the color resides in the fibroin or sericin. These methods are not very practical and are therefore not applicable for the large-scale production. Thus, instead of dyeing cocoons after their formation, it has been attempted to obtain a cocoon which has already been dyed at the time of its formation. For example, in EP 0 705 922 A1 it is described to coat with a dyestuff solution the spiracles of each grown larva, which is from the fourth diapauses to the fifth instar, several times so that the dyestuff is adsorbed on the silk glands within the larva. The larva is then allowed to secrete a silk filament from the spinneret thereof, whereby a colored cocoon is produced. However, this method requires many production steps and in addition a considerable time and labor are necessary for each production step. In JP 11-235136 silkworms are radiated with sun light intermittently in a predetermined manner, wherein coloring matter solution is given into the stigmas of the silkworms. The coloring matter is taken into the silk glands, and threads whose fibroin is colored are ejected from the silkworm. In CN1430904A commercially available food coloring has been used as additive in silkworm feed. Both in JP 11-235136 and CN1430904A colored cocoons are obtained wherein the dye is present in sericin so that the color fastness is not very good.

It is therefore an object of the present invention to overcome the above disadvantages and to provide an improved method for preparing a coloured silk material wherein the silk properties are maintained and color fastness is improved.

### SUMMARY OF THE INVENTION

In a first aspect the invention provides a method for producing intrinsically colored, luminescent silk fibroin. In the method the silkworms are fed with a luminescent dye. The obtained silk is degummed to obtain the colored silk fibroin.

The luminescent dye may be selected from the group consisting of xanthenes derivatives, cyanine derivatives, napththalene derivatives, coumarin derivatives, oxadiazole derivatives, pyrene derivatives, oxazine derivatives, acridine derivatives, arylmethine derivatives and tetrapyrrole derivatives, such as the group consisting of a compound according to

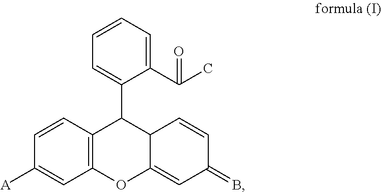

a compound according to

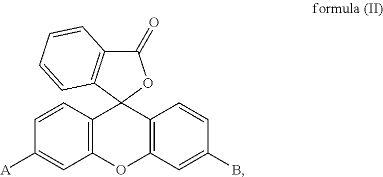

rhodamine 101, rhodamine 110, rhodamine 116, rhodamine B, rhodamine B base, and acridine orange. In each of formula (I) and formula (II), A may be independently an electron donating group selected from the group consisting of OH, an optionally substituted C₁₋₁₅ alkoxy group and NR₁R₂, or a polymer; whereas B may be independently selected from OH and NR₁R₂. Also, C may be selected from the group consisting of OH, an optionally substituted C₁₋₁₅ alkoxy group, halogen and NR₁R₂; and R₁ and R₂ may be independently selected from H, an optionally substituted C₁₋₁₀ alkyl and an optionally substituted C₁₋₁₅ alkoxy group. In case R₁ and R₂ in A and B are independently one of H, CH₃ or C₂H₅, then C may not be OH, OCH₃ or OC₂H₅.

In a second aspect the invention provides an intrinsically colored, luminescent silk fibroin obtainable by a method of the invention.

In a third aspect the invention provides the use of the intrinsically colored, luminescent silk fibroin to produce threads, yams or fabrics.

In a fourth aspect the invention provides the use of intrinsically colored, luminescent silk fibroin for biomedical applications.

In a fifth aspect, the invention provides a textile material intrinsically colored, luminescent silk fibroin obtainable by a method of the invention.

The invention will be better understood with reference to the detailed description when considered in conjunction with the accompanying drawings with the help of the figures illustrated in the attached drawings.

### BRIEF DESCRIPTION OF THE DRAWINGS

FIG. 1 depicts the schematic design of molecular structures for effective uptake into silk fibroin and incorporation of functional moieties for biological as well as textile applications.

FIG. 2 depicts (a) the 5th instar silkworms that have been fed with modified feed containing rhodamine B dye, and (b) the silk gland of such a silkworm.

FIG. 3 depicts (a) the colored cocoon produced by a 5th instar silkworm that has been fed with modified feed containing rhodamine B dye, and (b) the same cocoon under UV-light excitation.

FIG. 4 depicts the absorption colors and luminescent colors of the cocoons produced from silkworms fed with the modified feed containing rhodamine-based dyes.

FIG. 5 depicts the emission spectra of the raw silk produced by silkworms that have been fed with modified feed containing rhodamine B dye.

FIG. 6 depicts SEM micrographs of (a) non-modified raw silk and (b) colored silk produced by silkworms that have been fed the modified feed containing rhodamine B.

FIG. 7 depicts confocal images of (a) non-modified raw silk and (b) colored silk produced by silkworms that have been fed the modified feed containing rhodamine B.

FIG. 8 depicts confocal images and micrographs of degummed silk after treatment with (a) Marseille soap, (b) papain enzyme, and (c) savinase enzyme+triton X.

FIG. 9 depicts (a) the silk fibroins under LTV excitation and (b) the silk fibroin under 488 nm laser excitation.

FIG. 10 depicts the chemical structures as well as the corresponding release profiles of different dyes with balanced hydrophobic/hydrophilic properties.

FIG. 11 depicts the emission spectra of (a) Rhodamine B solutions with different pH values (b) Rhodamine B-incorporated colored silk fibroin after soaking in acid and in base.

FIG. 12 depicts the quantity of various dyes in sericin and fibroin per gram of raw silk as a function of their n-octanol/water partition coefficient (log p). The amount of each dye was normalized as micromole per gram of silk cocoon. a: fluorescein, b: sulforhodamine 101, c: Rhodamine 116, d: Rhodamine 110, e: acridine orange, f: Rhodamine 101, and g: Rhodamine B.

### DETAILED DESCRIPTION OF THE INVENTION

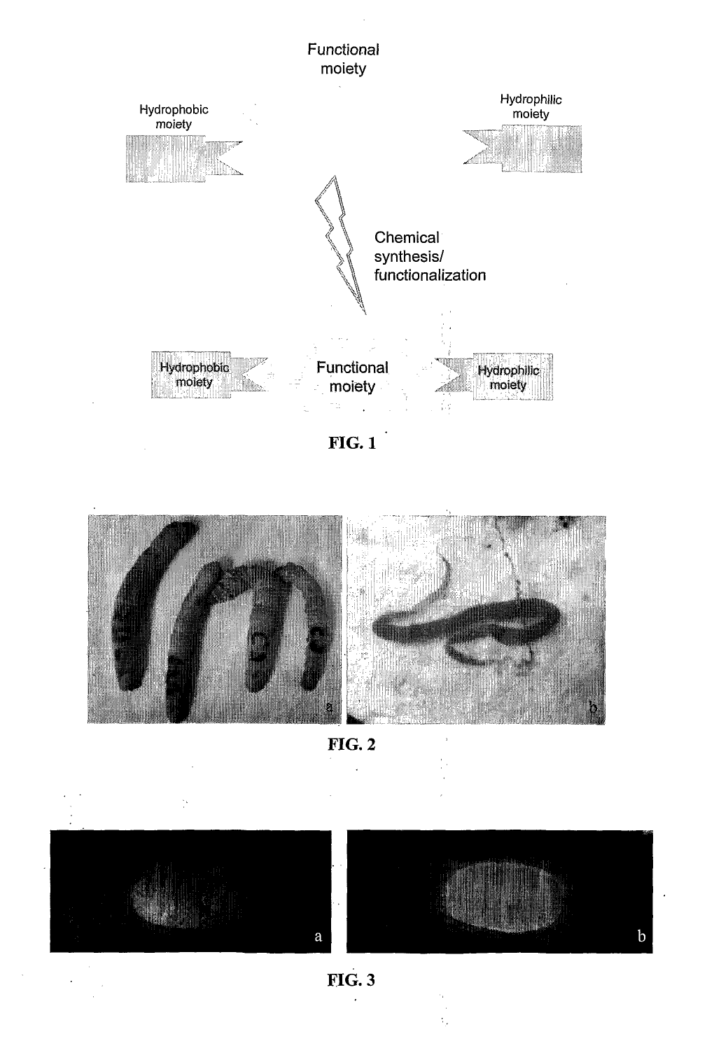

This present invention discloses the use, development, optimization and design of luminescent and functional molecular organic dyes with balanced hydrophobic/hydrophilic properties that can be effectively absorbed into silk glands of the silkworm. Specifically, zwitterionic and amphiphilic dye molecules may be used for this purpose. The schematic design of the functionalization of the amphiphilic dyes is illustrated in FIG. 1. These organic dye molecules are fed to the silkworm which incorporates the dyes directly into the silk fibers when producing them. The silk produced by using the molecular organic dye of the present invention obtains its original properties and has excellent color fastness to light, washing and the like. Further, the obtained silk may also be used for biomedical applications.

Thus, the present invention is the first demonstration of intrinsically colored, luminescent silk fibroin that is directly produced by silkworms. This represents a unique combination of know-how in molecular design with material characterization and application. By incorporating functional materials directly into the fibroin, there is minimum detrimental effect on the original properties of the silk. Further, due to the use of luminescent dyes, convenient use of confocal microscope to efficiently study their absorption is possible. These and other advantages will become more readily apparent from the following explanations and the appended drawings.

The amphiphilic organic dye molecule of the invention may be chosen from any dye molecule that may be absorbable into silk glands of the silkworm and that may be capable of directly dyeing the silk produced in the silkworm. The dye molecule of the invention may be a luminescent dye. The dye molecule may have amphiphilic character, i.e. it may be a chemical compound possessing both hydrophilic (water-loving) and lipophilic (fat-liking) properties. As shown in FIG. 1, said property may be achieved by functionalization of a basic functional moiety.

In one embodiment of the invention the luminescent dye may be selected from the group consisting of xanthenes derivatives, cyanine derivatives, napththalene derivativces, coumarin derivatives, oxadiazole derivatives, pyrene derivatives, oxazine derivatives, acridine derivatives, arylmethine derivatives and tetrapyrrole derivatives. In the above listing, xanthenes derivatives may be, but are not limited to, fluorescein (CAS number: 2321-07-5), rhodamines (cf., for example, below), Oregon green 488 (CAS number: 195136-58-4), ethyl eosin (CAS number: 6359-05-3), eosin Y (CAS number: 17372-87-1) texas red (CAS number: 199745-67-0 or 187099-99-6), and so on; cyanine derivatives may be, but not limited to, cyanine, indocarbocyanine, oxacarbocyanine (CAS number: 53213-82-4), thiacarbocyanine, merocyanine (CAS number: 62796-23-0), and so on; naphthalene derivatives may be, but not limited to, dansyl (CAS number: 10121-91-2), prodan derivatives, and so on; oxadiazole derivatives may be, but not limited to, pyridyloxazole (CAS number: 70380-75-5), nitrobenzoxadiazole (CAS number: 33984-50-8), benzoxadiazole (CAS number: 81377-14-2), and so on; pyrene derivatives may be, but not limited to, cascade blue (CAS numbers: 1325-87-7; 12238-23-2; 61725-40-4); and so on; oxazine derivatives may be, but not limited to, Nile red (CAS number: 7385-67-3), Nile blue (CAS number: 2381-85-3), cresyl violet (CAS number: 52659-20-8), oxazine 170 (CAS number: 62669-60-7), and so on; acridine derivatives may be, but not limited to, proflavin (CAS number: 92-62-6), acridine orange (CAS number: 260-94-6), acridine yellow (CAS number: 135-49-9), and so on; acrylmethine derivatives may be, but not limited to, auramine (CAS number: 2465-27-2), crystal violet (CAS number: 548-62-9), malachite green (CAS number: 569-64-2), and so on; tetrapyrrole derivatives may be, but not limited to, porphin (CAS number: 101-60-0), phthalocyanine (CAS number: 574-93-6), bilirubin (CAS number: 635-65-4) and so on. The above given CAS numbers are only exemplary and further derivatives of the compounds may form part of the present invention.

In one embodiment of the present invention, the amphiphilic organic dye may be selected from compounds according to general

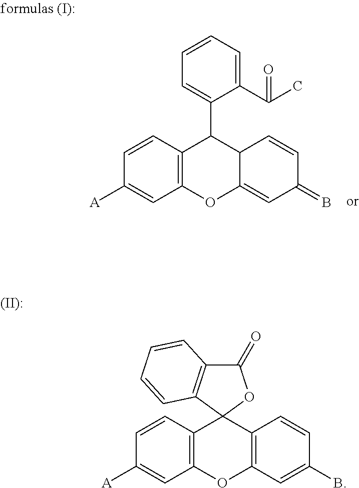

In each of formula (I) and (II) A may be independently an electron donating group selected from the group consisting of OH, an optionally substituted C₁₋₁₅ alkoxy group, NR₁R₂, and a polymer; B may be independently selected from OH and NR₁R₂; C may be selected from the group consisting of OH, an optionally substituted C₁₋₁₅ alkoxy group, halogen and NR₁R₂; and R₁ and R₂ are independently selected from H, an optionally substituted C₁₋₁₀ alkyl and an optionally substituted C₁₋₁₅ alkoxy group.

The term “alkyl”, alone or in combination, refers to a fully saturated aliphatic hydrocarbon. In certain embodiments, alkyls are optionally substituted. In certain embodiments, an alkyl comprises 1 to 10 carbon atoms, for example 1 to 8 carbon atoms or 1 to 6 carbon atoms, wherein (whenever it appears herein in any of the definitions given below) a numerical range, such as “1 to 10” or “C₁-C₁₀”, refers to each integer in the given range, e.g. “C₁-C₁₀ alkyl” means that an alkyl group comprises only 1 carbon atom, or 2 carbon atoms, 3 carbon atoms, 4 carbon atoms, 5 carbon atoms, 6 carbon atoms, 7 carbon atoms, 8 carbon atoms, 9 carbon atoms, up to and including 10 carbon atoms. Examples of alkyl groups include, but are not limited to, methyl, ethyl, n-propyl, isopropyl, n-butyl, isobutyl, sec-butyl, tert-butyl, tert-amyl, pentyl, hexyl, heptyl, octyl, nonyl, decyl and the like.

The term “alkoxy”, alone or in combination, refers to an aliphatic hydrocarbon having an alkyl-O-moiety. The alkoxy group may have 1 to 15 carbon atoms, such as 1 to 10 carbon atoms or 1 to 6 carbon atoms, for example 1, 2, 3, 4, 5, 6, 7, 8, 9, 10, 11, 12, 13, 14 or 15 carbon atoms. In certain embodiments, alkoxy groups are optionally substituted. Examples of alkoxy groups include, but are not limited to, methoxy, ethoxy, propoxy, butoxy, pentoxy and the like.

The term “optionally substituted” refers to a group in which none, one, or more than one of the hydrogen atoms has been replaced with one or more group(s) are independently selected from: alkyl, heteroalkyl, haloalkyl, heterohaloalkyl, cycloalkyl, aryl, arylalkyl, heteroaryl, non-aromatic heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups. In embodiments in which two or more hydrogen atoms have been substituted, the substituent groups may be linked to form a ring.

In one embodiment of the invention, A may be selected from OH, OCH₃, OCH₂CH₃, (OCH₂CH₂)ₓCH₃, NH₂, NHCH₃, N(CH₃)₂, NHC₂H₅, N(C₂H₅)₂, N(C₃H₇)₂, N(C₄H₉)₂, and N(C₅H₁₁)₂, wherein x is an integer between 1 and 6, such as 1, 2, 3, 4, 5 or 6. Also, B may be selected from OH, NH₂, NHCH₃, N(CH₃)₂, NHC₂H₅, N(C₂H₅)₂, N(C₃H₇)₂, N(C₄H₉)₂, and N(C₅H₁₁)₂, wherein x is an integer between 1 and 6, such as 1, 2, 3, 4, 5 or 6. C may be selected from the group consisting of OH, OCH₃, OC₂H₅ and OC₃H₇. In a still further embodiment of the present invention A and B may be independently selected from N(C₂H₅)₂, N(C₃H₇)₂, N(C₄H₉)₂, and N(C₅H₁₁)₂, and C may be OH.

The polymer which may be linked to the (for example, amphiphilic) organic dye as substituent A may be any polymer which may be suitably for the purpose of aiding the characteristics of the inventive dye. For example, the polymer may be chosen to improve the stability of the dye in the silk, such as to improve the stability in fibroin. A respective polymer may for example be a homopolymer or a copolymer. The polymer may in some embodiments be a linear, i.e. straight, polymer. In some embodiments it may be a hyperbranched polymer. The polymer will usually be selected to have a molecular weight in the range from about 500-1000 000, such as about 500-500.000, 500-200.000, 500-100.000, 500-50.000, 500-25.000, 500-10.000 or 500-5.000. The polymer may be, but is not limited to, polyaniline, polypyrrole and polythiophene, poly(ethylene glycol), polyglycolic acid, polycaprolactone, polylactic acid, polyhydroxyalkanoate, polyesters, polyanhydrides, polyorthoesters, polyphosphazenes, polyphosphates, polyphosphoesters, polyphosphonates, polydioxanones, polyhydroxyalkanoates, polycarbonates, polyalkylcarbonates, polyorthocabonates, polyesteramides, polyamides, polyamines, polypeptides, polyurethanes, polyetheresters, polyacrylates or combinations thereof. In one embodiment of the invention the polymer may be selected from the group consisting of poly(methylmetacrylate), poly(N-vinylimidazole), poly(hydroxyethyl methacrylate), poly(methyl methacrylate), poly(hydroxyethyl methacrylate), poly(ethoxy ethyl methacrylate), poly(acrylamide), poly(ethylene glycol), poly(lactic acid), poly(glycolic acid), gelatin and chitosan. The polymers may be incorporated in order to enhance absorption and retention in silk fibroin.

According to one embodiment of the present invention, in case of A and B R₁ and R₂ are independently one of H, NH₂, CH₃ or C₂H₅, then C is not OH, OCH₃ or OC₂H₅. For example, the following compounds are excluded from formulas (I) and (II): rhodamine 101, rhodamine 110, rhodamine 116, rhodamine 123, rhodamine 800, rhodamine B, rhodamine B base and rhodamine 6G.

In a further embodiment of the present invention, in the amphiphilic organic dye according to formulas (I) and (II) A may be independently an electron donating group selected from the group consisting of OH, an optionally substituted C₁₋₁₅ alkoxy group, NR₁R₂, or a polymer; B may be independently selected from OH and NR₁R₂; C may be selected from the group consisting of OH, an optionally substituted C₁₋₁₅ alkoxy group, halogen and NR₁R₂; and R₁ and R₂ may be independently selected from an optionally substituted C₃₋₁₀ alkyl and an optionally substituted C₁₋₁₅ alkoxy group. For example, A may be selected from OH, OCH₃, OCH₂CH₃, (OCH₂CH₂)ₓCH₃, NH₂, NHCH₃, N(CH₃)₂, NHC₂H₅, N(CH₂CH₃)₂, wherein x is an integer between 1 and 6, such as 1, 2, 3, 4, 5 or 6. Also, B may be selected from OH, NH₂, NHCH₃, N(CH₃)₂, NHC₂H₅, N(CH₂CH₃)₂, wherein x is an integer between 1 and 6, such as 1, 2, 3, 4, 5 or 6. C may be selected from the group consisting of OH, OCH₃, OC₂H₅ and OC₃H₇.

In case the organic dyes according to formulas (I) and (II) bear a positive charge, a counter ion will be present in order to have a neutral molecule. Suitable counter ions may be, but are not limited to, chloride, bromide, iodide, sulfate, hydrogensulfate, amiosulfate, methosulfate, ethosulfate, perchlorate, methylsulfonate, benzenesulfonate, methylbenzenesulfonate, oxalate, maleate, formate, acetate, hydroxyacetate, methoxyacetate, propionate, succinimide and tartrate, or the respective protonated form in case one proton from the basic organic dye is transferred to the counter ion.

Organic dye molecules of the invention may be prepared by general synthetic routes known in the art. For example, by contacting a compound of formula A

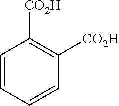

with a condensing agent (such as, but not limited to, sulfuric acid, hydrochloric acid or alkoxides) and at least two independently selected compounds of Formula B

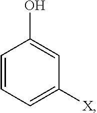

a compound according to general Formula (II) may be prepared. In the above Formula B X may be independently selected from OH, an optionally substituted C₁₋₁₅ alkoxy group and NR₁R₂. The compounds of Formula (II) may be transferred to compounds of Formula (I) by generally known methods. The preparation of such compounds of organic dye molecules according to Formula (I) or (II) is not limited to the above illustrative example. Further examples of possible preparation procedures are described in WO 2005/007678 or EP 0 468 821, the disclosure of which is incorporated by reference herein.

In the present invention a functional molecular organic dye with balanced hydrophobic/hydrophilic properties may be used as a feeding additive for silkworms. The additive may be used in any form which is suitable for the uptake into the silkworm. For example, the organic dye may be mixed in artificial silkworm feed or fresh mulberry leaves. The organic dye may be mixed in the feed by directly spraying or mixing them with the feed or by preparing solutions of the organic dye and spraying or coating such solutions onto the feed. Any kind of solvent may be used for preparing such solutions, as long as the solvent is not toxic to silkworms and as long as the organic dye is sufficiently soluble therein. Examples of such suitable solvents for preparing respective solutions include, but are not limited to, water (for example, regular drinking water, filtered water, deionized water) methanol, ethanol or mixtures thereof. The silkworm ingests the organic dye and as a consequence of that the silk gland also absorbed the dye and become colored. Then, the silkworm may start producing intrinsically colored and fluorescent silk cocoons (cf. FIG. 3). The functional molecular organic dye may be any of the above-mentioned luminescent compounds. In one embodiment of the invention the functional organic dye molecule may be, but not limited to, a compound according to formula (I), formula (II) or a compound such as, but not limited to, rhodamine 101 (for example CAS number: 41175-43-3, the CAS number depends on the anion), rhodamine 110 (for example CAS number: 13558-31-1), rhodamine 116 (for example CAS number 62669-77-6), rhodamine B (for example CAS number: 81-88-9), rhodamine B base (for example CAS number: 509-34-2) and acridine orange (for example CAS number: 260-94-6). The compounds may be used alone or in combinations with other compounds.

Thus, the present invention also refers to a method of producing an intrinsically colored, luminescent organic silk fibroin. The organic dye of the invention may be used to produce luminescent silk through the feeding method. Thereby, the organic dye is not only taken up by the silk gland of the silkworm but specifically is incorporated into the silk fibroin of the silk. Raw silk is comprised of fibroin and sericin. Up to now pigments are only present in sericin which is, however, removed during the processing (degumming). Thus, the coloring methods so far are not practical for actual applications. Now, the organic dye of the present invention is incorporated into the fibroin and thus color fastness is highly improved, wherein the general properties of the silk fibers are maintained. Surprisingly, the class of material described in the present invention has suitable properties for being taken up into silk gland and silk fibroin to produce intrinsically colored and luminescent silk fibroin.

In the present invention the method of producing intrinsically colored, luminescent silk fibroin first encompasses feeding silkworms with a feed comprising a luminescent dye as disclosed above. In one embodiment of the invention the luminescent dye may be a compound selected from an amphiphilic organic dye such as, a compound according to formula (I), formula (II), rhodamine 101, rhodamine 110, rhodamine 116, rhodamine B, rhodamine B base and acridine orange. As described above, feeding of the silkworms with the organic dyes of the present invention may be achieved by several ways depending on, for example, the feed or the form of the dye.

After the cocoon of the silk worm is formed and the cocoon is treated with hot air, steam, or boiling water, the obtained raw silk secreted from the silkworm fed is degummed. Degumming is the process of removing the sericin, or silk gum, from silk. Removing the gum improves the sheen, color, hand, and texture of the silk. Generally, the degumming process is not limited to a particular form or a particular process. Any process known in the prior art may be used to achieve the desired result. A degumming agent may be a soap, in particular an alkali-free soap, or any other compound generally used for this purpose. For example, the degumming agent may be, but not limited to, Marseille soap, papain and a bacterial protease. Suitable bacterial protease include the class of enzymes know as savinases. This class of enzyme is commercially used as a detergent protease and include the respective protease isolated from Bacillus clausii (that is commercialised by Novozymes) or from Bacillus Lentii (Swiss Prot accession number P29600).

The degumming agent may be used in concentrations generally used in the field of silk preparation. For example, the concentration may be, but not limited to, about 0.01 to about 2 wt %, such as about 0.01 to about 1.5 wt %, about 0.01 to about 1.0 wt % or 0.05 to 1 wt % based on the total weight of the degumming system. For example, the concentration of the degumming agent may be 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3; 1.4, 1.5, 1.6, 1.7, 1.8, 1.9 or 2.0 wt %. In view of the use of the soap, the degumming procedure is carried out in solutions at a pH between about 6 and about 11, for example between about 6 and about 10.0 or about 6 and about 9. The pH value of the solution may be 6.0, 6.5, 7.0, 7.5, 8.0, 8.5, 9.0, 9.5, 10, 10.5 or 11. The degumming is further carried out at a temperature between about 50 to about 100° C., such as about 50 to about 90° C., about 50 to about 80° C., about 55 to about 80° C. or about 55 to about 70° C. For example, the temperature of the degumming process may be 50° C., 55° C., 60° C., 65° C., 70° C., 75° C., 80° C., 85° C., 90° C., 95° C. or 100° C. The degumming process may be carried out for about 20 minutes, such as about 30 minutes, about 45 minutes or about 1 hour or even longer. The obtained colored silk fibroin may be further processed to obtain the final desired silk material (e.g. twisted etc.). In a further embodiment of the present invention the degumming reaction is carried out at a temperature of about 55-60° C. for 1 hour in case 0.1 wt % savinase is used as degumming agent. However, it is noted that all degumming agents may be used within the above given temperature ranges and in the above given amounts.

By imparting color and function to silk fibroin that is directly secreted by the silkworms, the present invention may result in significant cost saving and numerous new functions of colored fabric and biocompatible silk-based materials.

Thus, the functional silk may also be used for biomedical applications. Functional silk may be biocompatible and value-added. For example, silk fibroin may be used as suture threads or in applications in the tissue-engineering field as a scaffold support for the growth of artificial tissues such as bone and cartilage. Small molecule additives such as, for example, dyes (luminescent, photochromic, thermochromic, pH-sensitive) and drugs can be incorporated within the silk. Confocal microscopy can, for example, be used to efficiently study the absorption of these luminescent dyes into silk. Silk may be used as such or may be treated so that it delivers a drug. Attachment of the drug to the fabric can be covalent, or covalent via degradable bonds, or by any sort of binding (e.g. charge attraction) or absorption. Any drug can be potentially used; non-limiting examples of drugs include antibiotics, growth factors such as bone morphogenic proteins (BMPs) or growth differentiation factors (GDFs), growth inhibitors, chemo-attractants, and nucleic acids for transformation, with or without encapsulating materials. Further, sustained release of substances may also be possible as silk fibroin holds great promise for controlled drug delivery due to its unique structure and crystallinity properties as well as the other advantages discussed above. Silk microspheres can be fabricated using physical methods such as spray-drying. Fluorescence imaging, sensing of pH, temperature, and light could be achieved. Potential products may comprise, but are not limited to, luminescent silk, colored silk, fluorescent tissue-engineering scaffolds, sutures, fabrics for wound dressing.

The functional silk may also be used for textile applications. Dyes with modified structures could be developed for various luminescent color dyes. In addition, these dyes could be grafted to organic or inorganic polymers for improved color fastness. Compared to conventional dyeing, the quality of the color could be improved without detrimental effect to fabric's texture and sheen. Potential products may comprise, but are not limited to, intrinsically colored and luminescent silk yarns and fabrics. Thus, with the silk according to the present invention the dyeing step for silk fabrics can be eliminated and a more uniform color having less defect can be achieved. With the inventive silk, minimal changes to existing facilities or machineries must be made.

In order that the invention may be readily understood and put into practical effect, particular embodiments will now be described by way of the following non-limiting examples. It is understood that modification of detail may be made without departing from the scope of the invention.

### EXEMPLARY EMBODIMENTS OF THE INVENTION

Exemplary embodiments of methods according to the invention as well as reactants and further processes that may be used are shown in the following section and the appending figures.

### Example 1

### Method of Producing Intrinsically Colored, Luminescent Silk Fibroin by Feeding the Silkworm with Feed Comprising the Rhodamine B

0.05-0.1 wt % of rhodamine B (obtained as all other chemicals from Sigma Aldrich, Saint Louis, Mo., USA) were dissolved in deionized water and mixed into artificial silkworm feed. The modified feed was then fed to silkworms starting on the third day of fifth instar. After around 6 hours, an obvious color change was observed throughout the silkworm's body as shown in FIG. 2. The silk gland, obtained through dissection, also absorbed the dye and became colored. At day 8-9 of fifth instar, the silkworms started producing intrinsically colored and fluorescent silk cocoon as shown in FIG. 3.

### Example 2

### Characterization of Intrinsically Dyed Silk

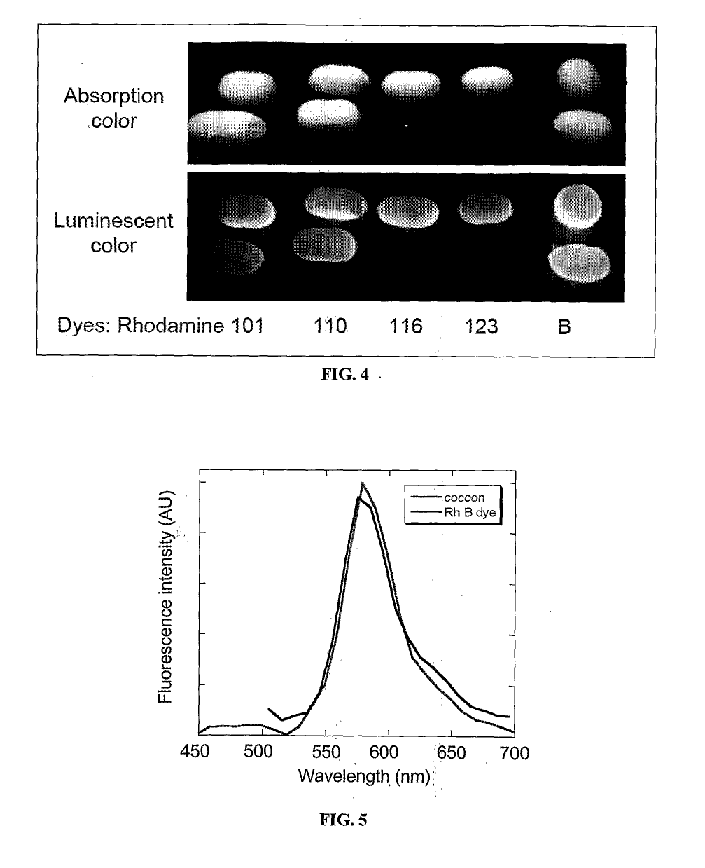

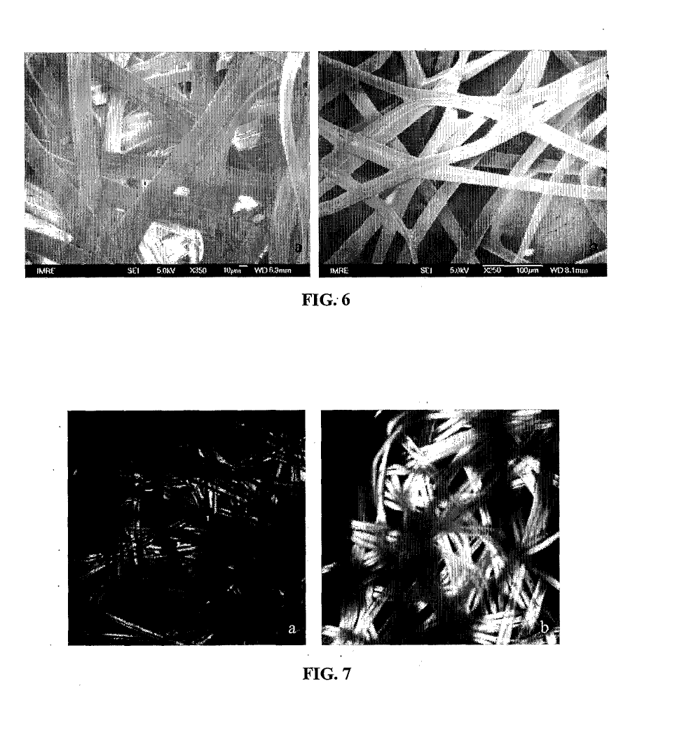

The presence of dyes in the raw silk could be observed even at the cocoon stage as shown in FIG. 4. It is further verified using fluorometer, where the emission peaks rhodamine B dyes were observed at 581 nm as shown in FIG. 5. The colored raw silk was imaged using field-emission scanning electron microscope (FESEM) and laser-scanning spectral confocal microscope. The FESEM micrograph displayed two fibroins held together by a layer of sericin. The diameter of each fibroin is 12±2 μm, while the thickness of sericin is around 2 μm. No noticeable morphological difference was observed between the colored raw silk and non-modified control silk as shown in FIG. 6.

Confocal images of the same set of sample are displayed in FIG. 7. The control cocoon showed only auto-fluorescence under the laser excitation; while the intrinsically colored cocoon was highly fluorescent. While the FESEM micrographs showed that the two silk filaments inside the raw silk are in complete contact, the confocal images showed some space between them. This indicates that the sericin, or at least some part of it, was not fluorescent and was thus not observable under the laser excitation. On the other hand, the two fibroin filaments appeared as distinct, luminescent fibers with uniform distribution of dye molecules. These images clearly showed the uptake of fluorescent dyes into the silk fibroin.

### Example 3

### Further Processing and Degumming to Produce Silk Fibroin

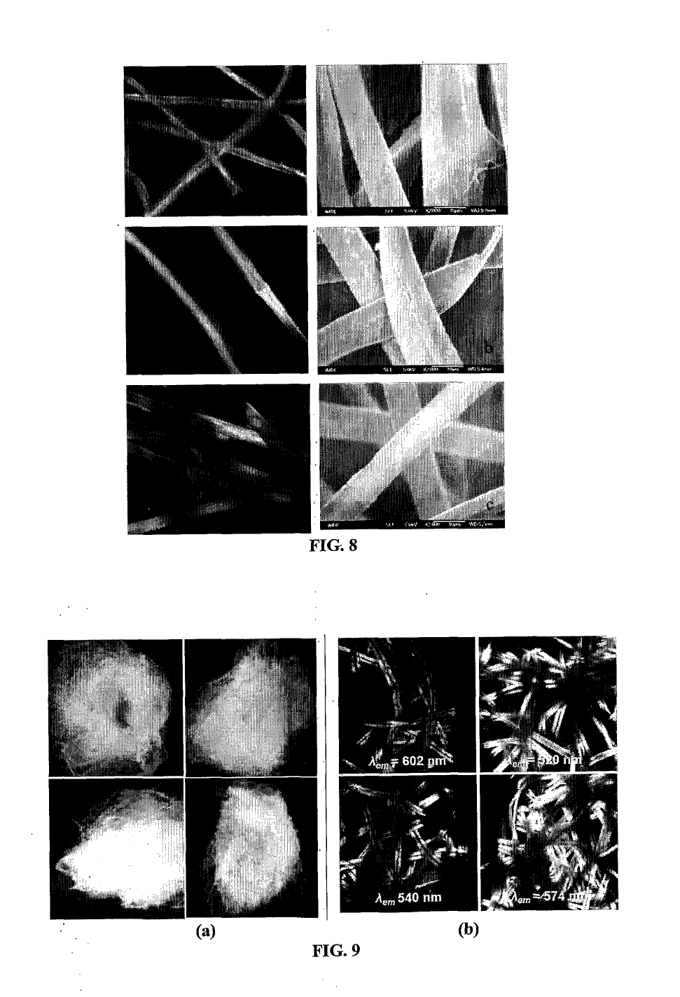

Further processing was carried out based largely on the industrial-standard procedure to convert the raw silk into fiber material for fabric. The cocoon was first stifled and dried at 110° C. for 1 h and 80° C. for 2 h, followed by immersion in a series of water baths at 95, 80, and 60° C. to loosen the sericin and aid in reeling. Throughout this process, very little color was lost and no noticeable decrease of color intensity was observed. The reeled silk was subsequently weighed and subjected to degumming process to remove the sericin layer, giving it the desired luster and touch. Various combinations of degumming agents, dispersants, and degumming conditions were explored with varying degrees of color retention as summarized in Table 1 and FIG. 8. The optimal degumming procedure with effective removal of sericin and highest dye retention was achieved using the Savinase enzyme (Novozymes A/S, Bagsvaerd, Denmark) at 65° C. and pH 9 for 1 hour.

TABLE 1
Processing parameters for the degumming step

| Degumming | Concentration | Temperature | | Duration |
|---|---|---|---|---|
| agent | (wt %) | (° C.) | pH | (hour) |
| Soap + Na₂CO₃ | 0.5 + 0.2 | 90 | 11 | 1 |
| Papain | 0.1 | 55 | 6 | 1 |
| Savinase | 0.1 | 55 | 9 | 1 |
| Savinase + triton X | 0.1/1 | 55 | 9 | 1 |

The successful uptake of dyes into the silk gland and its subsequent integration with silk fibroin may be attributed to the zwitterionic and amphiphilic nature of the rhodamine B. Compared to previous attempts using highly hydrophilic molecules, Table 2 illustrates that the balanced hydrophilic/hydrophobic nature of this particular dye may contribute to its more efficient uptake through hydrophobic channel in the silkworm body.

TABLE 2
Efficiency of uptake of the different dyes into the silkworm and the silk fibroin

| | | | | Counter | | | |
|---|---|---|---|---|---|---|---|
| | A | B | C | ion | in worm | in fibroin | charge |
| Rhodamine 110 | NH₂ | NH₂⁺ | OH | Cl⁻ | yes | yes | zwitter |
| Rhodamine B | N(CH₂CH₃)₂ | N(CH₂CH₃)₂ | OH | Cl⁻ | yes | yes | zwitter |
| Rhodamine 101 | N(quinlizn)₂ | N(quinlizn)₂ | OH | — | yes | yes | zwitter |
| Rhodamine 116 | NHCH₃ | NHCH₃ | OH | HClO₄ | yes | yes | zwitter |
| Rhodamine B | N(CH₂CH₃)₂₍ₐ₎ | N(CH₂CH₃)₂₍ₐ₎ | -(a) | — | yes | yes | neutral |
| base | | | | | | | |
| Rhodamine 123 | NH₂ | NH₂ | OCH₃ | HCl | no | no | cationic |
| Rhodamine 800 | N(quinlizn)₂(b) | N(quinlizn)₂(b) | -(b) | Cl⁻ | no | no | cationic |
| Rhodamine 6G | NHCH₂CH₃ | NCH₂CH₃ | OCH₂CH₃ | HCl | — | — | cationic |

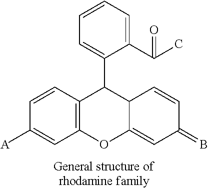

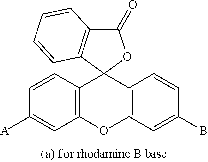

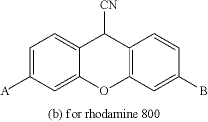

The colors of the silk fibroin could be clearly observed under UV excitation (FIG. 9a) as well as under 488 nm laser emission (FIG. 9b).

Apart from the rhodamine series, other dye molecules were also tested and while Acridine orange has also been found to have efficient uptake into silk gland and silk fluorescein, Alcian blue, Bromophenol blue did not display significant uptake into silk.

Thus, the desired dye molecules should ideally possess balanced hydrophobic/hydrophilic properties or be zwitterionic to ensure efficient uptake into silk gland and silk fibroin. These properties will be incorporated into the design of new molecules to produce functional silk through the feeding method.

### Example 4

### Release Profile of Dyes in Intrinsically Colored Silk

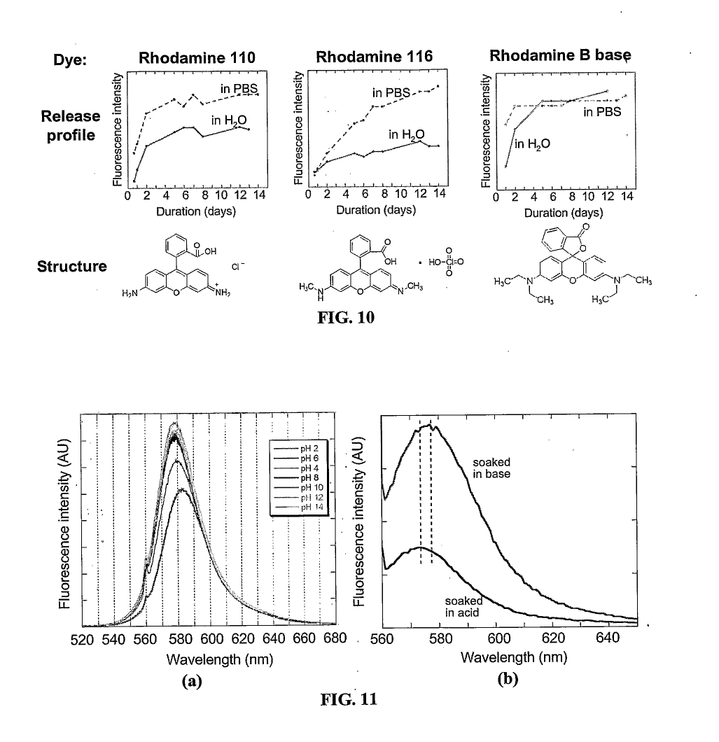

As illustrated in FIG. 10, the amount of dyes released generally increases with time, indicated by the higher fluorescence intensity of the solution. Thus, sustained release of the dye molecules from the intrinsically colored silk is possible. In addition, various release profiles can be obtained from different dyes. A model study on release profiles of drugs or other small molecules from silk biomaterials could be established by investigation of the release profiles of functionalized amphiphilic dye molecules whose rate and duration of release may be tuned by selection or modification of their molecular structures.

### Example 5

### pH Sensing Applications Of Intrinsically Colored Silk

FIG. 11a demonstrates that the fluorescence intensity increases while the emission wavelength is blue-shifted with increasing pH between pH 2 and 6 for the rhodamine B solution.

For silk samples containing Rhodamine B which are soaked in base, a higher luminescent intensity with an emission wavelength at 573 nm was observed in FIG. 11b. When a similar silk sample is soaked in acid solution, a lower luminescent intensity with an emission wavelength at 579 nm was observed.

This illustrates that the applications of the functional silk containing amphiphilic dye molecules may include but are not limited to wound dressing, to monitor, for example, the pH changes of the wounded skin.

### Example 6

### Quantity of Various Dyes in Sericin and Fibroin

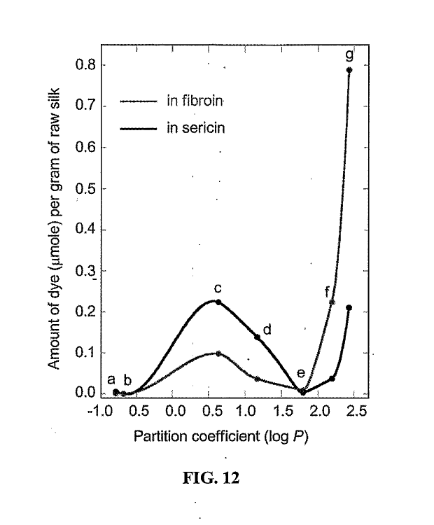

FIG. 12 summarizes the amounts of various dyes distributed in silk's fibroin and sericin as a function of log P, a measure of hydrophobicity. Fluorescein (log P=−0.79) and sulforhodamine 101 (−0.69) exhibited negligible presence in silk and in silkworm because of their rapid clearance out of the silkworm's body. With increased log P, Rhodamine 116 (0.64) and Rhodamine 110 (1.17) were found in substantial amounts in both sericin and fibroin, as well as in silkworm body. Rhodamine 116 (0.64) was found more in silk sericin than fibroin; similar to the case of naturally colored That golden silk (0.55) in which the natural, golden pigment was also found mostly on silk sericin. Rhodamine 110 with higher log P had a lower uptake into both hydrophilic sericin and hydrophobic fibroin. The amount of dye observed in sericin and fibroin decreased with a further increase of hydrophobicity. For example, acridine orange (1.8) has a very low concentration in silk. However, a sharp reversal of this trend was observed for Rhodamine 101 (2.19) and Rhodamine B (2.43), which were found at a much higher concentration with a majority residing in silk fibroin rather than sericin (e.g. 350 ppm for Rhodamine B). This indicates the presence of another factor, aside from hydrophobicity, that affects the uptake and distribution of substances in vivo.

The concentration of Rhodamine B in gland content just before the silk production reached ˜1 mM, as measured from silk fibroin. At this high concentration, dimer would be formed (cf. Kajiwara, T., Chambers R. W. & Kearns D. R. Dimer spectra of rhodamine B. Chem. Phys. Lett. 22, 37-40 (1973); Selwyn, J, E. & Steinfeld, J. I. Aggregation equilibria of xanthene dyes. J. Phys. Chem. 76, 762-774 (1972)). Molecular self-assembly/aggregation could expose either hydrophilic carboxylic acid or hydrophobic ethyl groups outward to change hydrophobicity of Rhodamine B dimers, resulting in more efficient transfer from gland walls to sericin and from sericin to fibroin to produce highly luminescent silk. In comparison, the concentration of acridine orange in gland content was very low. This is because the non-amphiphilic structure of acridine orange does not allow tuneable hydrophobicity upon formation of dimmers (cf. Antonov, L., Gergov, G., Petrov, V., Kubista, M. & Nygren, J. UV-Vis spectroscopic and chemometric study on the aggregation of ionic dyes in water. Talanta 49, 99-106 (1999)). Acridine orange molecules were thus retained in the gland walls with very low uptake into gland content.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognised that various modifications are possible within the scope of the invention claimed. Additional objects, advantages, and features of this invention will become apparent to those skilled in the art upon examination of the foregoing examples and the appended claims. Thus, it should be understood that although the present invention is specifically disclosed by exemplary embodiments and optional features, modification and variation of the inventions embodied therein herein disclosed may be resorted to by those skilled in the art, and that such modifications and variations are considered to be within the scope of this invention. In addition, where features or aspects of the invention are described in terms of Markush groups, those skilled in the art will recognise that the invention is also thereby described in terms of any individual member or subgroup of members of the Markush group.

1. A method of producing intrinsically colored, luminescent silk fibroin comprising:
feeding silkworms with an feed comprising a luminescent dye; and
degumming the obtained raw silk secreted from the silkworm fed.

2. The method of claim 1, wherein the luminescent dye is a zwitterionic or amphiphilic molecule.

3. The method of claim 1, wherein the luminescent dye is selected from the group consisting of xanthenes derivatives, cyanine derivatives, napththalene derivativces, coumarin derivatives, oxadiazole derivatives, pyrene derivatives, oxazine derivatives, acridine derivatives, arylmethine derivatives and tetrapyrrole derivatives.

4. The method of claim 1, wherein the luminescent dye is selected from the group consisting of a compound according to formula (I), 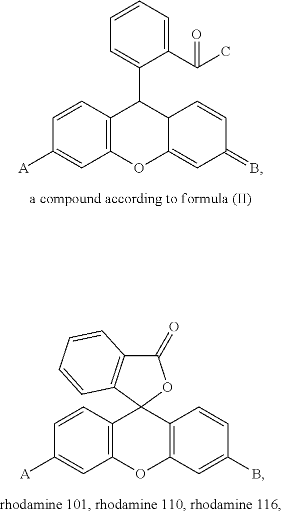
rhodamine B is independently selected from OH and NR1R2; C is selected from the group consisting of OH, an optionally substituted CI-15 alkoxy group, halogen and NR1R2; and
R1 and R2 are independently selected from an optionally substituted C3.16 alkyl and an optionally substituted C1-15 alkoxy group.

5. The method of claim 4, wherein A is selected from OH, OCH3, OCH2CH3, (OCH2CH2% CH3, NH2, NHCH3, N(CH3)2, NHC2H5, N(C2H5)2, N(C3H7)2, N(C4H9)2, and W51411)2, wherein x is an integer between 1 and 6.

6. The method of claim 4, wherein B is selected from OH, NH2, NHCH3, N(CH3)2, NHC2H5, N(C2Hsh, N(C3H7)2, N(C4H9)2, and N(C5H 02, wherein x is an integer between 1 and 6.

7. The method of claim 4, wherein C is selected from the group consisting of OH, OCH3, 0C2Hond 0C31-17.

8. The method of claim 4, wherein the polymer is selected from the group consisting of poly(methylmethacrylate), poly(N-vinylimidazole), poly(hydroxyethyl methacrylate), poly(methyl methacrylate), poly(hydroxyethyl methacrylate), poly(ethoxy ethyl methacrylate), poly(acrylamide), poly(ethylene glycol), poly(lactic acid), poly(glycolic acid), gelatine and chitosan.

9. The method of claim 4, wherein in case of A and B 125 and R2 are independently one of H, NH2, CH3 or C2H5, then C is not OH, OCH3 or 0C2H5.

10. The method of claim 1, wherein the degturuning agent is Marseille soap, papain, or a bacterial protease.

11. The method of claim 10, wherein the bacterial protease is a savinase.

12. The method of claim 1, wherein the degumming is carried out in solutions with pH between 6 and 11.

13. The method of claim 12, wherein the degumming is carried out in solutions with pH between 6.5 and 10.

14. The method of claim 1, wherein the degumming is carried out at a temperature between about 50° C. and 10p ° C.

15. The method of claim 14, wherein the degutnming is carried out at a temperature of about 55° C.

16. An intrinsically colored, luminescent silk fibroin obtainable by a method comprising:
feeding silkworms with an feed comprising a luminescent dye; and
degumming the obtained raw silk secreted from the silkworm fed.

17. A method comprising using an intrinsically colored, luminescent silk fibroin to produce threads, yams or fabrics, wherein the intrinsically colored, luminescent silk fibroin is obtained by a method comprising:
feeding silkworms with an feed comprising a luminescent dye; and
degumming the obtained raw silk secreted from the silkworm fed.

18. The use of A method comprising using an intrinsically colored, luminescent silk fibroin of claim 16 for biomedical applications, wherein the intrinsically colored, luminescent silk fibroin is obtained by a method comprising:
feeding silkworms with an feed comprising a luminescent dye; and
degumming the obtained raw silk secreted from the silkworm fed.

19. The method of claim 18, wherein the biomedical applications include sutures, fabrics for wound dressings, sustained release of substances, fluorescence imaging and pH sensing.

20. A textile material comprising intrinsically colored, luminescent silk fibroin obtainable by a method comprising:
feeding silkworms with an feed comprising a luminescent dye; and
degumming the obtained raw silk secreted from the silkworm fed.

21. The textile material of claim 20, wherein the material is a thread, a yarn or a fabric.
